(19) United States
(12) Patent Application Publication
(10) Pub. No.: US 2013/0151267 A1
(43) Pub. Date: Jun. 13, 2013

(54) SYSTEM TO ALLOW FOR A PHARMACIST TO STORE A FILLED PRESCRIPTION IN AN ELECTRONIC LOCKBOX FOR SUBSEQUENT RETRIEVAL BY A CUSTOMER AFTER IDENTITY VERIFICATION

(71) Applicant: P. Vincent Mehdizadeh, West Hills, CA (US)

(72) Inventor: P. Vincent Mehdizadeh, West Hills, CA (US)

(73) Assignee: PVM INTERNATIONAL, INC., West Hills, CA (US)

(21) Appl. No.: 13/314,692

(22) Filed: Dec. 8, 2011

(51) Int. Cl. G06Q 50/22

(52) U.S. Cl. 705/2

(57) ABSTRACT

A system that is used at pharmacies where the identity of the individual purchasing medication needs to be verified. The system incorporates magnetic swipe cards that have refillable monetary amounts that are deducted every time the patient is dispensed medication. The system also incorporates fingerprint verification and facial and retinal information to insure the correct patient's identity and whether the patient's authorization to acquire the medication has not expired. The prescription is placed in an electronic lockbox which opens when the individual has been properly identified.

### BACKGROUND OF THE INVENTION

1. Field of the Invention

The present invention relates to a computer system used in conjunction with electronically controlled lockboxes that are used by a pharmacist to store a customer's prescription medication for subsequent retrieval using biometrics in order to verify the customer's identity. The present invention relates to the problem of a patient desiring to pick up a pre-approved prescription medication from a pharmacy after the pharmacy has closed or after the pharmacy department of a drugstore has closed and the pharmacist is no longer at the pharmacy.

2. Description of the Prior Art

To the best of the present inventor's knowledge, there is no system comparable to the present invention which is available on the market.

### SUMMARY OF THE INVENTION

The present invention is a system that is used at pharmacies where the identity of the individual picking up a prescription medication refill needs to be verified. In one embodiment, the system incorporates magnetic swipe cards that have refillable monetary amounts that are deducted every time the patient picks up a refill prescription medication. The system also incorporates fingerprint verification, facial recognition and retinal recognition to insure the correct patient's identity as well as determine whether the patient's physician's prescription to acquire the prescription medication has not expired.

In general, a customer has a prescription called into a pharmacist from a doctor's office and if the customer is already in the system database, that customer can pick up the medication at anytime thereafter at the system upon notification by the pharmacy that the medicine is ready, via text message to a mobile phone, email, or automated phone call. If the customer is new to the system, they would have to bring a physical prescription slip and register with the initial visit.

The customer frequently has the prescription refilled on a periodic basis such as monthly. In general, it is necessary for the pharmacist to be present when handing the prescription medication to the customer. A problem arises when the customer has to work and cannot arrive at the pharmacy until some time after the pharmacy has closed. The present invention addresses refilling the prescription at a time after the pharmacy has closed and the pharmacist is not present to hand the prescription to the customer. This also assumes that the pharmacy does not deliver the prescription medication to the patient's home or place of business with signature required or the customer does not wish to pay for this extra service.

The present invention provides a software system to accumulate the number of prescription refills used by a customer over a period of time such as a month, two months, six months, etc. to determine if the customer is properly using the prescription to obtain only the prescribed amount of prescription medication allotted for a given period of time.

It is an object of the present invention to provide a point of pickup lockbox system so that if a customer orders a refill and cannot arrive at the pharmacy while it is open, a lockbox system is set up where the pharmacist can leave the filled prescription in an electronic lockbox for a patient to retrieve after the pharmacy operating hours.

It is another object of the present invention to provide a system which incorporates magnetic swipe cards that have refillable monetary amounts that are deducted every time the patient picks up the dispensed medication.

It is a further object of the present invention to provide a system which also incorporates information selected from the group consisting of fingerprint verification, facial verification, and retinal verification, to insure that the identity of the customer opening the electronic lockbox with the filled prescription is correct before the medication is dispensed to the patient.

It is an additional object of the present invention to allow retail operators to track inventory as well as demonstrate compliance with local and state laws regarding dispensing the prescription medication.

Therefore, it is an object of the present invention to provide a lockbox system to dispense the prescription medication refill after all appropriate verification steps have been taken. This includes biometric fingerprint verification, digital facial recognition photographs, computer facial recognition software and retinal recognition software, and additional information about the customer including age, height, weight, color of eyes, hair color, social security number, amount of refills left on the prescription and the authorized refill rate (monthly, quarterly, etc.).

The pharmacy can include any desired multiplicity of lockboxes such as 20, 50, 100, 200 etc. The size can be any size such as four (4) inches high by four (4) inches wide by two (2), four (4), six (6) or eight (8) inches deep. The prescription refill will be loaded in the lockbox, through the rear, by the pharmacist or other qualified pharmacy employee. When the customer arrives, the customer is identified by a digital photographic camera monitoring the lockboxes and utilizing a fingerprint verification device at the lockboxes to verify the customer's identity. A digital camera with facial recognition and retinal recognition software confirms the customer's identity in a computer database. The customer will then swipe the customer's magnetically encoded card containing other confidential customer information and the electronic lockbox door will electronically be opened. The customer then removes the prescription medication and closes the lockbox to reset the system. The swipe card may also have a money balance from which the price of the refill is deducted leaving a net balance for future prescription purchases.

The lock boxes will be at a secure location within the drugstore or at a secure location on the outside of the pharmacy with twenty-four hour video monitoring and direct communication to police authorities if someone tries to break into the lockbox.

Further novel features and other objects of the present invention will become apparent from the following detailed description, discussion and the appended claims, taken in conjunction with the drawings.

### BRIEF DESCRIPTION OF THE DRAWINGS

Referring particularly to the drawings for the purpose of illustration only and not limitation, there is illustrated:

FIG. 1 is a block diagram illustrating some of the steps in the present invention system at a pharmacy authorized to dispense prescription medications, including entering background information about the customer and the physician prescribing the medication;

FIG. 2 is a block diagram setting forth the personal information about the customer entered into a central database and entered into a swipe card used by the customer to unlock the electronic lockbox and retrieve the prescription refill;

FIG. 3 is an illustration of an electronic lockbox at a pharmacy with monitoring and customer verification at the location of the lockbox, also illustrating a customer at the lockbox;

FIG. 4 is an illustration of an electronic lockbox opened with a customer picking up the prescription medication; and

FIG. 5 is an illustration of the electronic lockbox closed after the prescription medication has been picked up by the customer.

### DETAILED DESCRIPTION OF THE PREFERRED EMBODIMENTS

Although specific embodiments of the present invention will now be described with reference to the drawings, it should be understood that such embodiments are by way of example only and merely illustrative of but a small number of the many possible specific embodiments which can represent applications of the principles of the present invention. Various changes and modifications obvious to one skilled in the art to which the present invention pertains are deemed to be within the spirit, scope and contemplation of the present invention as further defined in the appended claims.

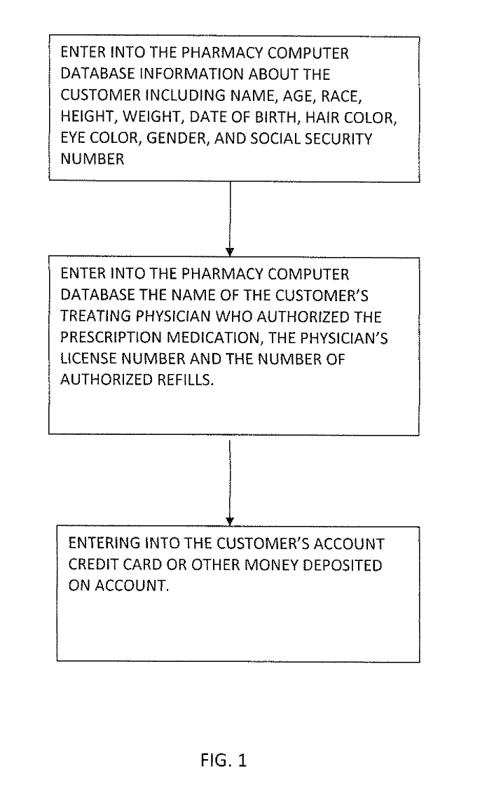

Referring to the first block step in FIG. 1, the first step in the present invention system is software which enables an employee of a pharmacy to register the individual who is a customer for the prescription medication into the pharmacy's establishment system using a computer based application that asks for and enters into the system specific information about the customer including the patient's name, date of birth, age, race. height, weight, hair color, eye color and social security number. The system also enters information about the physician who prescribed the medication including the medication prescribed, the patient's treating physician, the physician's license number and the expiration date of the prescription. In addition, the amount of money credited to the patient's card is also entered. As an example, a newly registered patient may choose to put $200.00 on the card.

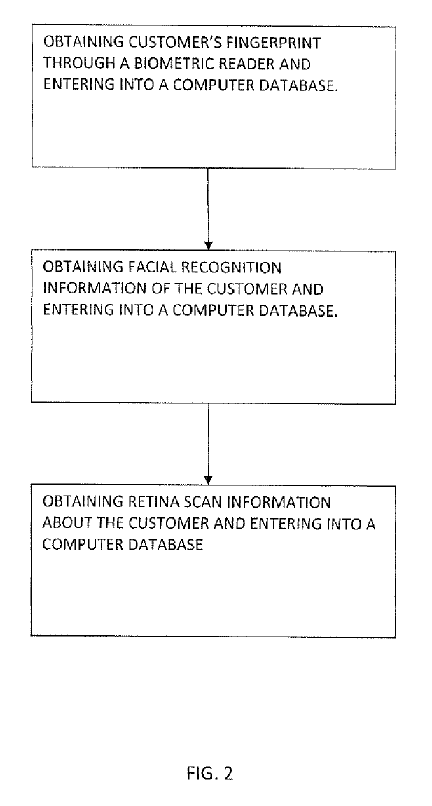

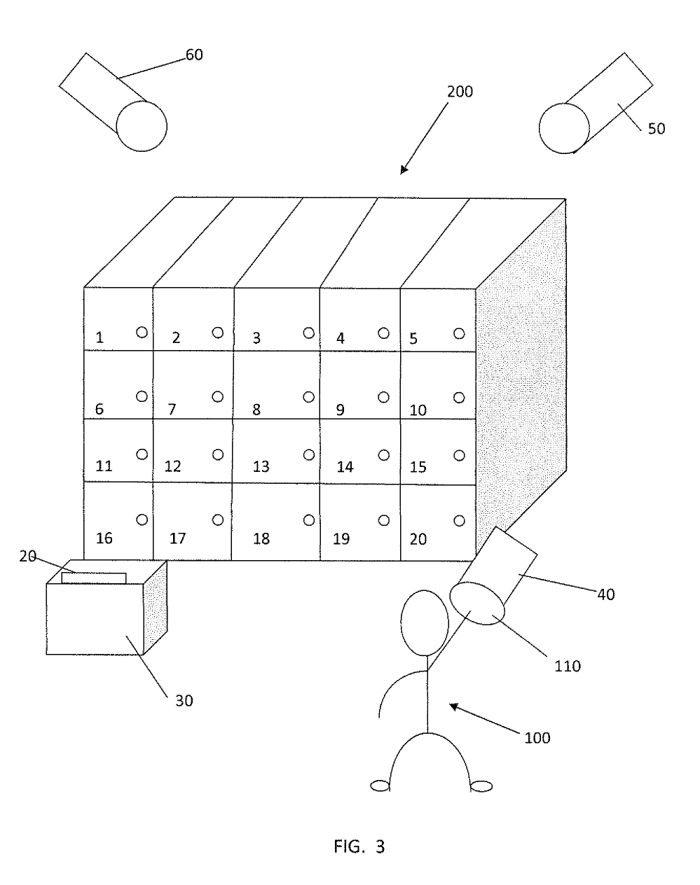

Referring to FIG. 2, once the patient is included in the database, fingerprint samples are taken. One method to take the patient's fingerprint is through a biometric reader that is plugged into a USB port of the computer. A separate USB port is utilized by a card reader that programs and encodes the patient's information and fingerprint sample on the registration card that is given to the patient. Facial recognition information and retinal recognition information are also obtained from the customer and entered into the computer database and onto the customer's swipe card.

A visual digital face recognition picture of the customer is taken to be utilized at the electronic lockbox location.

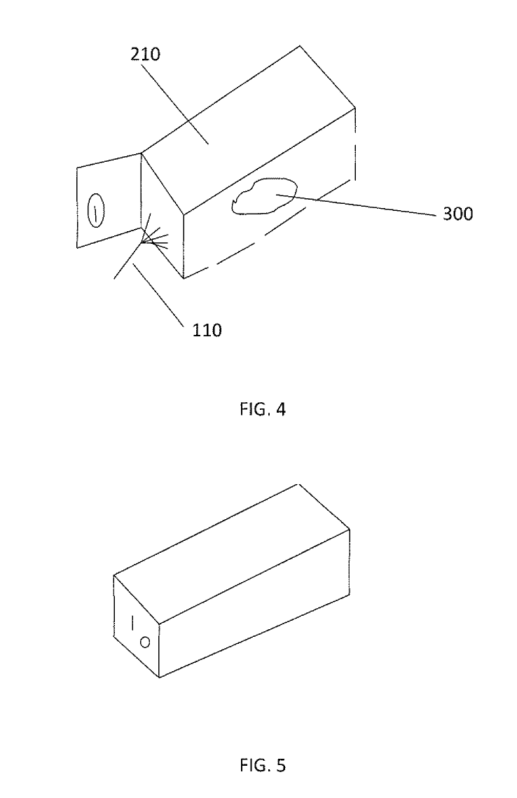

Referring to FIG. 4, the electronic lockbox 200 has individual lockboxes numbered 1 though 20. A card reader 30 is affixed to the electronic lockbox and the customer's card is swiped through the card reader 30. The fingerprint recognition device 40 is also attached to the lockbox 200 so the customer 100 places a finger into the device reader 40. A camera 50 with facial recognition and retinal recognition features further confirms the person's identity. The camera 50 can also be used for twenty-four hour visual monitoring of the lockbox area.

Once the customer or patient has the registration card, the patient may use the registration card at the lockbox location of the pharmacy by sliding the card through the card reader and placing the patient's finger on the biometric reader to confirm the patient's identity and standing in front of the digital face recognition reader to further confirm the customer's identity. Referring to FIG. 4, the prescription refill 300 has previously been placed in a selected electronic lockbox and the customer is advised as to which lockbox number contains the customer's prescription refill. After completing all of the above steps. the individual lockbox 210 is electronically opened by computer and the patent places his/her hand 110 into the lockbox 210 to pick up the prescription medication 300.

After retrieving the prescription refill, the customer closes the electronic lockbox as illustrated in FIG. 5 and this resets the system for further use of the same lockbox by another customer. The information about the purchase is sent to a central computer database of the pharmacy to monitor the refill and deduct the purchase price from the customer's credit card amount.

The lockbox is at a secure location within a drugstore adjacent the pharmacy or just exposed through the wall of a standalone pharmacy. A video camera 50 monitors the lockbox twenty-four (24) hours a day, seven (7) days a week. In this way, a customer can pick up the refill after the pharmacy has closed.

The prescription can also be a first-time prescription medication order in addition to a refill, but usually the person takes the prescription into the pharmacy for the first-time filling.

Of course the present invention is not intended to be restricted to any particular form or arrangement, or any specific embodiment, or any specific use, disclosed herein, since the same may be modified in various particulars or relations without departing from the spirit or scope of the claimed invention hereinabove shown and described of which the apparatus or method shown is intended only for illustration and disclosure of an operative embodiment and not to show all of the various forms or modifications in which this invention might be embodied or operated.

What is claimed is:

1. A system to dispense and monitor refills for a prescription medication by an individual, comprising:
a. utilizing a computer based application at a pharmacy authorized to fill and dispense the prescription medication to obtain information about an individual seeking to refill a prescription for a prescription medication, the information about the individual selected from the group consisting of the individual's name, date of birth, age, height, weight, hair color, eye color, and information about the physician issuing the prescription selected from the group consisting of the name of the physician, the physician's license number and the expiration date of the prescription;
b. obtaining payment for the prescription medication from the group consisting of obtaining an advance payment for prescription refills from the individual at registration through keeping a credit card and insurance information within the database, storing cash value on the registration card used to fill and refill the prescription medication, and allowing for payment options at the system itself at the point of prescription retrieval by the individual;
c. utilizing the computer based application to obtain the individual's fingerprint through a biometric reader that is plugged into a USB port of a computer;
d. utilizing a digital face recognition device to encode the customer's face and retinal information so that the customer making a pickup of a prescription refill at an electronic lockbox is viewed to confirm the customer's identity;
e. utilizing a separate USB port by a card reader that programs and encodes the individual's information and fingerprint sample on the registration card that is given to the individual;
f. a multiplicity of lockboxes having a fingerprint reader, a camera with facial and retinal recognition software, and a card reader with card swipe capability;
g. retrieval of prescription medication refills through an electronic lockbox system when an individual slides a registration card through a card reader and places a finger on the biometric reader and stands in front of the digital viewing camera to confirm the individual's identity prior to granting access to a particular electronic lockbox computer assigned to the individual for the prescription pickup and deducting payment from the individual's prepaid account.
h. the electronic lockbox is electronically opened by computer to enable the customer to retrieve the prescription medication refill placed in the assigned lockbox; and
i. the system is automatically reset when the assigned lockbox is closed.

2. A system comprising: utilizing an assigned electronic lockbox into which a prescription medication has been placed to dispense the prescription medication and transmit information to a central computer database describing in detail the amount of prescription medication dispensed, tracking a customer's use of a prescription medication and monitoring how often a refill of the prescription medication is requested by the customer as well as confirming that the customer to whom access to the electronic lockbox is granted which contains the prescription medication is in fact the correct customer who was issued the prescription.

3. A system to dispense and monitor fillings for prescription medication by an individual, comprising:
a. utilizing a computer based application at a pharmacy to fill and enable an individual to gain access to an assigned electronic lockbox containing the prescription medication and to obtain information about an individual seeking the prescription medication;
b. obtaining an advance payment for prescription refills from the individual and entering the advance payment on a registration card used to fill and refill the prescription medication;
c. utilizing the computer based application to obtain the individual's fingerprint and digital photographic facial image;
d. utilizing a computer application that programs and encodes the individual's personal information and fingerprint sample on a registration card that is given to the individual; and
e. dispensing a prescription medication through an electronically opened lockbox after the system has confirmed the individual's identity to obtain the medication, the lockbox being accessible after the pharmacy has closed.

4. The system in accordance with claim 3, further comprising:
a. utilizing the electronic lockbox system to confirm the individual's identity by the individual's fingerprint and inputting the amount charged to the individual's registration card and confirming the balance of refills and money left on the individual's registration card; and
b. utilizing the computer application to assemble records of prescriptions filled by each individual serviced by that pharmacy.

5. The system in accordance with claim 4, further comprising:
a. the individual's personal information is selected from the group consisting of the individual's name, date of birth, prescription information including the name of the individual's treating physician, the physician's license number and the expiration date of the prescription and amount of refills authorized.

6. The system in accordance with claim 5, further comprising:
a. utilizing the computer based application to obtain the individual's fingerprint through a biometric reader that is plugged into a USB port of a computer.

7. The system in accordance with claim 6, further comprising:
a. utilizing a separate USB port by a card reader that programs and encodes the individual's information and fingerprint sample on the registration card that is given to the individual.

8. The system in accordance with claim 7, further comprising:
a. allowing retrieval of a prescription medication by the individual when the individual slides the registration card through a card reader and places the individual's finger on the biometric reader to confirm the individual's identity. and inputting the amount of prescription medication purchased and the amount charged to the individual's registration card which is automatically deducted from the patient's registration card.

9. The system in accordance with claim 3, further comprising twenty-four hour video viewing of the lockbox area.
